(19) United States
(12) Patent Application Publication
(10) Pub. No.: US 2024/0285793 A1
(43) Pub. Date: Aug. 29, 2024

(54) TOBACCO MOSAIC VIRUS DELIVERY OF MITOXANTRONE FOR CANCER THERAPY

(71) Applicant: CASE WESTERN RESERVE UNIVERSITY, Cleveland, OH (US)

(72) Inventor: Nicole F. Steinmetz, San Diego, CA (US)

(21) Appl. No.: 18/657,338

(22) Filed: May 7, 2024

Related U.S. Application Data
(63) Continuation-in-part of application No. 16/597,509, filed on Oct. 9, 2019, now abandoned.
(63) Continuation-in-part of application No. 18/426,706, filed on Jan. 30, 2024.
Application No. 18/426,706 is a continuation of application No. 16/854,444, filed on Apr. 21, 2020, now Pat. No. 11,883,505.
Application No. 16/854,444 is a continuation of application No. 15/741,017, filed on Dec. 29, 2017, now Pat. No. 10,624,975.
(60) Provisional application No. 62/743,319, filed on Oct. 9, 2018.
(60) Provisional application No. 62/185,881, filed on Jun. 29, 2015.
(60) Provisional application No. 62/201,227, filed on Aug. 5, 2015.

(51) Int. Cl. A61K 47/69; A61K 9/00; A61K 9/51; A61K 31/555; A61K 39/00; A61K 39/12; A61P 35/00

(52) U.S. Cl. CPC A61K 47/6901; A61K 9/0019; A61K 9/5184; A61K 31/555; A61K 39/12; A61P 35/00; A61K 2039/585; C12N 2750/12022; C12N 2750/12023; C12N 2770/00034; C12N 2770/00042; C12N 2770/32022; C12N 2770/32023

(57) ABSTRACT

A method of treating cancer in a subject that includes administering to the cancer a therapeutically effective amount of an anti-cancer virus particle, the virus particle including a rod-shaped plant virus or virus-like particle and mitoxantrone (MTO) or analogs thereof, wherein the MTO is loaded into the interior channel of the rod-shaped plant virus particle.

### RELATED APPLICATION

This application is a Continuation-in-part of U.S. application Ser. No. 16/597,509, filed Oct. 9, 2019, which claims priority from U.S. Provisional Application No. 62/743,319, filed Oct. 9, 2018. This application is also a Continuation-in-Part of U.S. application Ser. No. 18/426,706, filed Jan. 30, 2024, which is continuation of U.S. application Ser. No. 16/854,444, filed Apr. 21, 2020 (now U.S. Pat. No. 11,883,505), which is a continuation of U.S. application Ser. No. 15/741,017, filed Dec. 29, 2017 (now U.S. Pat. No. 10,624,975) which is a national phase filing of PCT/US2016/039961, filed Jun. 29, 2016, which claims priority to U.S. Application No. 62/185,881, filed Jun. 29, 2015, and 62/201,227, filed Aug. 5, 2015. All subject matter of which are incorporated herein by reference in their entirety.

### BACKGROUND

Mitoxantrone (MTO) is a clinically approved chemotherapeutic used to treat metastatic breast cancer, advanced prostate cancer, as well as forms of leukemia and lymphoma. Mitoxantrone is a type II topoisomerase inhibitor, and as such interferes with the cell cycle leading to apoptosis by disrupting DNA synthesis and DNA repair in both healthy and cancer cells by intercalation between DNA bases. MTO has been used to treat forms of cancer either as a solo chemotherapy regimen or as component in cocktail treatments. However, MTO is a highly potent poison that while effective against tumor cells, also causes systemic toxicities thus limiting its dose, particularly to the heart. For example, it has been shown that dose-dependent cardiotoxicity of MTO results in reduction of left ventricular ejection fraction along with congestive heart failure, thereby greatly limiting the clinical utility of MTO.

Plant-virus based-nanotechnologies provide an exciting alternative to the more traditional and more frequently exploited synthetic nanoparticles. Plant viruses, or viruses in general, can be considered as nature's delivery vehicles; viruses are designed to penetrate cells and deliver cargo. While mammalian viruses have been used to deliver genes for nucleic acid therapy, plant viruses offer a safer alternative due to their inability to infect or replicate in mammalian cells. Like other biologics, plant virus-based nanoparticles can be manufactured through a variety of homologous and heterologous expression systems at high yields and with high quality control and assurance. Plant viruses are monodisperse and many of their structures are known to near atomic resolution; therefore enabling structure-based design of high precision nanodrug delivery systems.

### SUMMARY

Embodiments described herein relate to an anti-cancer virus particle. The anti-cancer virus particle includes a rod-shaped plant virus particle and mitoxantrone (MTO) or an analog thereof, wherein the MTO is loaded into an interior channel of the rod-shaped plant virus particle. In some embodiments, the MTO or analog thereof is non-covalently loaded into the interior channel of the rod-shaped plant virus particle. In some embodiments, the MTO analog selected from the group consisting of pixantrone and losoxantrone.

In some embodiments, the rod-shaped plant virus particle is a member of the Virgaviridae family. In some embodiments, the rod-shaped plant virus particle is a tobacco mosaic virus (TMV).

In some embodiments, the exterior surface of the rod-shaped plant virus particle is PEGylated. In some embodiments, a targeting ligand is attached to the exterior of the rod-shaped plant virus particle.

Other embodiments described herein relate to methods of treating cancer in a subject. The method includes administering to the subject a therapeutically effective amount of an anti-cancer virus particle. The virus particle includes a rod-shaped plant virus or virus-like particle and mitoxantrone (MTO) or an analog thereof. The MTO is loaded into the interior channel of the rod-shaped plant virus particle. In some embodiments, the anti-cancer virus particle is administered together with a pharmaceutically acceptable carrier.

In some embodiments, release of the MTO or an analog thereof from the rod-shaped plant virus particle is pH dependent. In some embodiments, the release is triggered by an acidic tumor microenvironment.

In some embodiments, the cancer is selected from the group consisting of breast cancer, prostate cancer, fibrosarcoma, leukemia and lymphoma. In some embodiments, the breast cancer is a metastatic breast cancer. The metastatic breast cancer can include triple negative breast cancer.

The method can further include administering a therapeutically effective amount of an additional anticancer agent or therapy to the subject. The additional anti-cancer agent can include an antitumor agent. In some embodiments, the additional anti-cancer agent can be selected from the group consisting of doxorubicin, vincristine, and prednisone. The additional anticancer therapy can include radiation and/or ablation therapy.

In some embodiments, the anti-cancer virus particle is administered to a tumor site in the subject. In some embodiments, the anti-cancer virus particle is administered to the subject systemically. In some embodiments, the subject has a history of cardiac disease and/or one or more cardiac events. In some embodiments, the anti-cancer virus particle is administered at an amount effective to reduce or limit MTO-associated cardiotoxicity in the subject.

Additional embodiments described herein relate to methods of treating triple negative breast cancer in a subject. The method includes administering to the subject a therapeutically effective amount of an anti-cancer virus particle. The virus particle includes a rod-shaped plant virus or virus-like particle and mitoxantrone (MTO) or an analog thereof. The MTO is loaded into the interior channel of the rod-shaped plant virus particle. In some embodiments, the anti-cancer virus particle is administered together with a pharmaceutically acceptable carrier.

In some embodiments, release of the MTO or an analog thereof from the rod-shaped plant virus particle is pH dependent. In some embodiments, the release is triggered by an acidic tumor microenvironment.

The method can further include administering a therapeutically effective amount of an additional anticancer agent or therapy to the subject. The additional anti-cancer agent can include an antitumor agent. In some embodiments, the additional anti-cancer agent can be selected from the group consisting of doxorubicin, vincristine, and prednisone. The additional anticancer therapy can include radiation and/or ablation therapy.

In some embodiments, the anti-cancer virus particle is administered to a tumor site in the subject. In some embodiments, the anti-cancer virus particle is administered to the subject systemically.

### BRIEF DESCRIPTION OF THE FIGURES

FIG. 1 is an illustration showing mitoxantrone (MTO) loading into TMV. TMV forms a 300×18 nm nanorod (left), with a 4 mm-wide channel lined with glutamic acids. The negative charge of the glutamic acids allows for electrostatic interactions with the positively charged MTO, allowing for pH dependent drug-loading and -release.

FIGS. 2(A-D) are graphical illustrations showing that MTO-TMV particle characterization. FPLC using a Superose6 column and ÄKTO purifier (A, detectors: 260 nm for TMV's protein, and 622 nm for MTO) and TEM of negative-stained samples (B) were used to confirm _{MTO}TMV particle integrity. Drug loading was determined using UV-Vis spectroscopy (C), with absorbance peaks at 260 and 280 nm corresponding to TMV and 622 nm corresponding to MTO. Drug release (D) was performed via dialysis in varying buffer conditions, with samples removed at designated time-points for analysis and quantification of MTO content per TMV by UV-vis.

FIGS. 3(A-D) illustrate cell viability as a function of MTO concentration comparing (A) free MTO and MTO-TMV using the MTT assay. IC₅₀ values of free MTO and MTO-TMV against MDA-MB-231, HT1080, and PC3 cells. (B) Cellular uptake of free MTO and MTO-TMV was quantified by flow cytometry (FACS). Histograms are shown in (C) and corresponding mean fluorescence intensities in (D). Stats: Experiments were done in triplicates and repeated at least twice; mean values and standard deviations are shown.

FIG. 4 illustrates MTO-TMV vs. free MTO treatment using a mouse model of triple negative breast cancer (MDA-MB-231 s.c. xenografts in NRCnu/nu mice, n=5). Treatments (PBS, TMV, MTO, MTO-TMV) were given on days 1, 5, 10 at a dosage of 1 mg kg-1 MTO; TMV was normalized to the equivalent amount of TMV in _{MTO}TMV.

### DETAILED DESCRIPTION

Methods involving conventional molecular biology techniques are described herein. Such techniques are generally known in the art and are described in detail in methodology treatises, such as Current Protocols in Molecular Biology, ed. Ausubel et al., Greene Publishing and Wiley-Interscience, New York, 1992 (with periodic updates). Unless otherwise defined, all technical terms used herein have the same meaning as commonly understood by one of ordinary skill in the art to which the application pertains. Commonly understood definitions of molecular biology terms can be found in, for example, Rieger et al., Glossary of Genetics: Classical and Molecular, 5th Edition, Springer-Verlag: New York, 1991, and Lewin, Genes V, Oxford University Press: New York, 1994.

Unless otherwise defined, all technical and scientific terms used herein have the same meaning as commonly understood by one of ordinary skill in the art to which this invention belongs. The terminology used in the description of the invention herein is for describing particular embodiments only and is not intended to be limiting of the invention. All publications, patent applications, patents, and other references mentioned herein are incorporated by reference in their entirety.

As used in the description of the invention and the appended claims, the singular forms “a,” “an,” and “the” are intended to include the plural forms as well, unless the context clearly indicates otherwise. In addition, the recitations of numerical ranges by endpoints include all numbers subsumed within that range (e.g., 1 to 5 includes 1, 1.5, 2, 2.75, 3, 3.80, 4, 5, etc.).

The terms “comprise,” “comprising,” “include,” “including,” “have,” and “having” are used in the inclusive, open sense, meaning that additional elements may be included. The terms “such as”, “e.g.”, as used herein are non-limiting and are for illustrative purposes only. “Including” and “including but not limited to” are used interchangeably.

The term “or” as used herein should be understood to mean “and/or”, unless the context clearly indicates otherwise.

The terms “cancer” or “tumor” refer to any neoplastic growth in a subject, including an initial tumor and any metastases. The cancer can be of the liquid or solid tumor type. Liquid tumors include tumors of hematological origin, including, e.g., myelomas (e.g., multiple myeloma), leukemias (e.g., Waldenstrom's syndrome, chronic lymphocytic leukemia, other leukemias), and lymphomas (e.g., B-cell lymphomas, non-Hodgkin's lymphoma). Solid tumors can originate in organs and include cancers of the lungs, brain, breasts, prostate, ovaries, colon, kidneys and liver.

The terms “cancer cell” or “tumor cell” can refer to cells that divide at an abnormal (i.e., increased) rate. Cancer cells include, but are not limited to, carcinomas, such as squamous cell carcinoma, non-small cell carcinoma (e.g., non-small cell lung carcinoma), small cell carcinoma (e.g., small cell lung carcinoma), basal cell carcinoma, sweat gland carcinoma, sebaceous gland carcinoma, adenocarcinoma, papillary carcinoma, papillary adenocarcinoma, cystadenocarcinoma, medullary carcinoma, undifferentiated carcinoma, bronchogenic carcinoma, melanoma, renal cell carcinoma, hepatoma-liver cell carcinoma, bile duct carcinoma, cholangiocarcinoma, papillary carcinoma, transitional cell carcinoma, choriocarcinoma, semonoma, embryonal carcinoma, mammary carcinomas, gastrointestinal carcinoma, colonic carcinomas, bladder carcinoma, prostate carcinoma, and squamous cell carcinoma of the neck and head region; sarcomas, such as fibrosarcoma, myxosarcoma, liposarcoma, chondrosarcoma, osteogenic sarcoma, chordosarcoma, angiosarcoma, endotheliosarcoma, lymphangiosarcoma, synoviosarcoma and mesotheliosarcoma; hematologic cancers, such as myelomas, leukemias (e.g., acute myelogenous leukemia, chronic lymphocytic leukemia, granulocytic leukemia, monocytic leukemia, lymphocytic leukemia), lymphomas (e.g., follicular lymphoma, mantle cell lymphoma, diffuse large B-cell lymphoma, malignant lymphoma, plasmocytoma, reticulum cell sarcoma, or Hodgkin's disease), and tumors of the nervous system including glioma, glioblastoma multiform, meningoma, medulloblastoma, schwannoma and epidymoma.

The term “nanoparticle” refers to any particle having a diameter of less than 1000 nanometers (nm). In general, the nanoparticles should have dimensions small enough to allow their uptake by eukaryotic cells. Typically, the nanoparticles have a longest straight dimension (e.g., diameter) of 200 nm or less. In some embodiments, the nanoparticles have a diameter of 100 nm or less. Smaller nanoparticles, e.g., having diameters of 50 nm or less, e.g., about 1 nm to about 30 nm or about 1 nm to about 5 nm, are used in some embodiments.

The phrases “parenteral administration” and “administered parenterally” are art-recognized terms and include modes of administration other than enteral and topical administration, such as injections, and include, without limitation, intratumoral, intravenous, intramuscular, intrapleural, intravascular, intrapericardial, intraarterial, intrathecal, intracapsular, intraorbital, intracardiac, intradermal, intraperitoneal, transtracheal, subcutaneous, subcuticular, intra-articular, subcapsular, subarachnoid, intraspinal and intrasternal injection and infusion.

The phrases “systemic administration,” “administered systemically,” “peripheral administration” and “administered peripherally” as used herein mean the administration of a compound, agent or other material other than directly into a specific tissue, organ, or region of the subject being treated (e.g., tumor site), such that it enters the animal's system and, thus, is subject to metabolism and other like processes, for example, subcutaneous administration.

“Treating”, as used herein, means ameliorating the effects of, or delaying, halting or reversing the progress of a disease or disorder. The word encompasses reducing the severity of a symptom of a disease or disorder and/or the frequency of a symptom of a disease or disorder.

A “subject”, as used therein, can be a human or non-human animal. Non-human animals include, for example, livestock and pets, such as ovine, bovine, porcine, canine, feline and murine mammals, as well as reptiles, birds and fish. Preferably, the subject is human.

The language “effective amount” or “therapeutically effective amount” refers to a sufficient amount of the composition used in the practice of the invention that is effective to provide effective treatment in a subject, depending on the compound being used. That result can be reduction and/or alleviation of the signs, symptoms, or causes of a disease or disorder, or any other desired alteration of a biological system. An appropriate therapeutic amount in any individual case may be determined by one of ordinary skill in the art using routine experimentation.

A “prophylactic” or “preventive” treatment is a treatment administered to a subject who does not exhibit signs of a disease or disorder, or exhibits only early signs of the disease or disorder, for the purpose of decreasing the risk of developing pathology associated with the disease or disorder.

A “therapeutic” treatment is a treatment administered to a subject who exhibits signs of pathology of a disease or disorder for the purpose of diminishing or eliminating those signs.

“Pharmaceutically acceptable carrier” refers herein to a composition suitable for delivering an active pharmaceutical ingredient, such as the composition of the present invention, to a subject without excessive toxicity or other complications while maintaining the biological activity of the active pharmaceutical ingredient. Protein-stabilizing excipients, such as mannitol, sucrose, polysorbate-80 and phosphate buffers, are typically found in such carriers, although the carriers should not be construed as being limited only to these compounds.

Throughout the description, where compositions are described as having, including, or comprising, specific components, it is contemplated that compositions also consist essentially of, or consist of, the recited components. Similarly, where methods or processes are described as having, including, or comprising specific process steps, the processes also consist essentially of, or consist of, the recited processing steps. Further, it should be understood that the order of steps or order for performing certain actions is immaterial so long as the compositions and methods described herein remains operable. Moreover, two or more steps or actions can be conducted simultaneously.

Embodiments described herein relate to an anti-cancer virus particle. The virus particle includes a rod-shaped plant virus or virus-like particle (VLPs) and mitoxantrone (MTO) or an analog thereof, wherein the MTO is loaded into an interior channel of the rod-shaped plant virus particle. Using both in vitro and in vivo studies, it has been shown that uptake of MTO loaded rod-shaped virus particles by cancer cells has a cytotoxic effect on cancer cells as a solo therapy. For example, using a mouse model of triple negative breast cancer MTO loaded rod-shaped virus particles were shown to outperform the cytotoxic tumor reducing effect of free MTO while reducing the severe cardiac side effects commonly associated with MTO treatment. It has also been shown that rod-shaped nanoparticles loaded with MTO do not accumulate in the heart following administration to a subject. Thus, it is contemplated that rod-shaped plant virus nanoparticle delivery of MTO can overcome cardiotoxicity while enhancing drug delivery thereby improving the overall treatment efficacy for subjects in need thereof.

The rod-shaped virus particles or virus-like particle can be nonreplicating and noninfectious in the subject to avoid infection of the subject and can be regarded as safe from a human health and agricultural perspective. In planta production prevents endotoxin contamination that may be a byproduct of other virus or virus-like particle systems derived from E. coli. The virus particles or virus-like particles are scalable, stable over a range of temperatures (4-60° C.) and solvent:buffer mixtures.

In some embodiments, rod-shaped plant virus particles or virus-like particles in which the viral nucleic acid is not present are loaded with MTO or an analog thereof and are administered to the subject. Virus-like particles lacking their nucleic acid are non-replicating and non-infectious regardless of the subject into which they are introduced.

In other embodiments, the rod-shaped plant virus particles include a nucleic acid within the virus particle. If present, the nucleic acid will typically be the nucleic acid encoding the virus. However, in some embodiments the viral nucleic acid may have been replaced with exogenous nucleic acid. In some embodiments, the nucleic acid is RNA, while in other embodiments the nucleic acid is DNA. A virus particle including nucleic acid will still be nonreplicating and noninfectious when it is introduced into a subject which it cannot infect. For example, plant virus particles will typically be nonreplicating and noninfectious when introduced into an animal subject.

A rod-shaped plant virus is a virus that primarily infects plants, is non-enveloped, and is shaped as a rigid helical rod with a helical symmetry. Rod shaped viruses also include a central canal where MTO or an analog thereof can be loaded into. Rod-shaped plant virus particles are distinguished from filamentous plant virus particles as a result of being inflexible, shorter, and thicker in diameter. For example, Virgaviridae have a length of about 200 to about 400 nm, and a diameter of about 15-25 nm. Virgaviridae have other characteristics, such as having a single-stranded RNA positive sense genome with a 3′-tRNA like structure and no polyA tail, and coat proteins of 19-24 kilodaltons.

In some embodiments, the rod-shaped plant virus belongs to a specific virus family, genus, or species. For example, in some embodiments, the rod-shaped plant virus belongs to the Virgaviridae family. The Virgaviridae family includes the genus Furovirus, Hordevirus, Pecluvirus, Pomovirus, Tobamovirus, and Tobravirus. In some embodiments, the rod-shaped plant virus belongs to the genus Tobamovirus. In further embodiments, the rod-shaped plant virus belongs to the tobacco mosaic virus (TMV) species. TMV has a capsid made from 2130 molecules of coat protein and one molecule of genomic single strand RNA 6400 bases long. The coat protein self-assembles into the rod like helical structure (16.3 proteins per helix turn) around the RNA which forms a hairpin loop structure. The protein monomer consists of 158 amino acids which are assembled into four main alpha-helices, which are joined by a prominent loop proximal to the axis of the virion. Virions are ˜300 nm in length and ˜18 nm in diameter. Negatively stained electron microphotographs show a distinct inner channel of ˜4 nm.

TMV is a high aspect ratio soft nanotube, a hollow cylinder with exterior dimensions of 300×18 nm and 4 nm wide channel. In some embodiments, TMV can include one or more point mutations. In particular embodiments, TMV can include a T158K point mutation. TMV can be produced by mechanical inoculation of Nicotiana benthamiana plants using well known protocols.

The surface chemistry of the interior and exterior surfaces of a rod-shaped plant virus particle or virus like particle are distinct and charge-driven drug loading mechanisms that can be used to accommodate therapeutics inside the channel for drug delivery. Without being bound by theory, drug loading into a rod-shaped plant virus or virus like particle, such as loading MTO into TMV virus particles (referred to herein as MTO-TMV) is believed to be driven by a combination of charge interactions and hydrophobic stacking. In some embodiments, the MTO or an analog thereof can be also be bound to the exterior surface of the particles through non-specific drug-protein interactions.

In some embodiments, MTO or an analog thereof is loaded into an interior channel of the rod-shaped virus particle or virus-like particle. In some embodiments, an analog of mitoxantrone, or a mix of mitoxantrone and one or more analog thereof, can be loaded into a rod-shaped plant virus as described herein. Analogs of mitoxantrone for use in a composition or method described herein can include, but are not limited to, a hetero-analog of mitoxantrone. Exemplary analogs of mitoxantrone include pixantrone and losoxantrone (biantrazole).

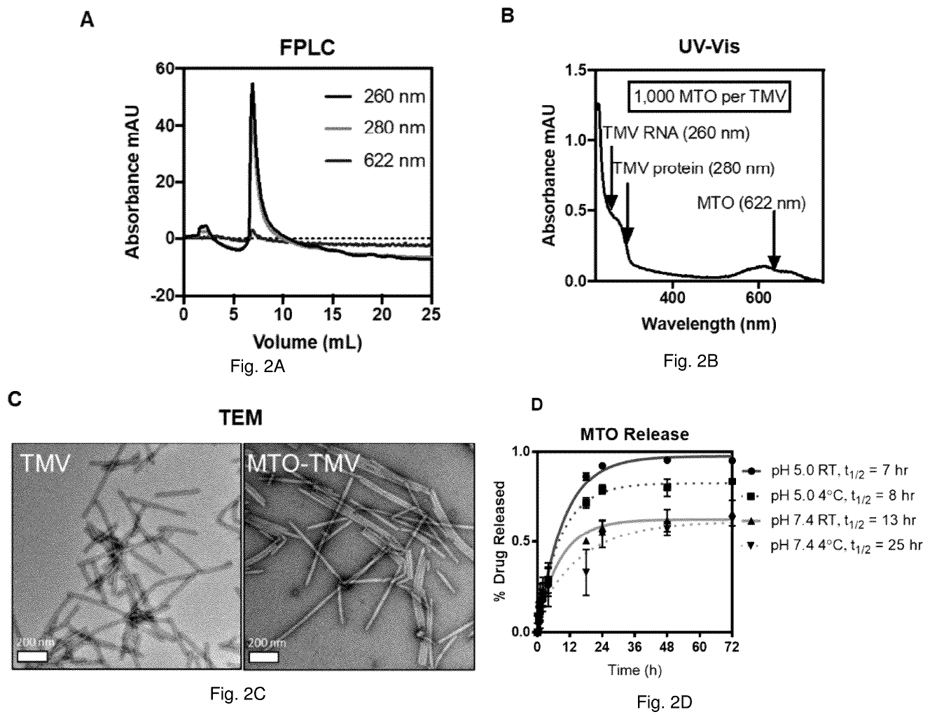

In an exemplary embodiment, TMV can be mixed with 10,000-fold molar excess of mitoxantrone in a potassium phosphate buffer for about 18 hours. The reaction mix can then be purified via centrifugation to remove excess free MTO. A resulting MTO-TMV pellet can be resuspended and further purified by centrifugation, and/or elution techniques. Samples can be further analyzed using UV-visable spectroscopy to determine concentration and transmission election microscopy (TEM) and size exclusion chromatography (SEC) to confirm particle monodispersity and integrity as well as to assess drug loading and release (see FIG. 2). In some embodiments, loading of MTO or an analog thereof into a rod-shaped plant virus or virus-like particle can yield about 1000 MTO per TMV particle carrier.

Alternately, rather than being non-covalently loaded into the virus particle, the MTO or analog thereof can bond or be conjugated to an interior surface of a rod-shaped plant or virus-like particle. The term “conjugate” when made in reference to a cargo molecule, such as MTO or an analog thereof, and a rod-shaped plant virus particle as used herein, means covalently linking a cargo molecule to a virus subject to the single limitation that the nature and size of the agent and the site at which it is covalently linked to the virus particle does not interfere with the biodistribution of the modified virus.

In general, MTO or an analog thereof, can be conjugated to the plant virus by any suitable technique, with appropriate consideration of the need for pharmacokinetic stability and reduced overall toxicity to the patient. The MTO or an analog thereof can be linked to the interior channel or the exterior of the virus, while in some embodiments the MTO or an analog thereof is linked to both the interior and the exterior of the virus. The location of the MTO or an analog thereof on the interior or exterior can be governed by the amino acids of the viral coat protein.

MTO or an analog thereof can be coupled to a rod-shaped virus particle or virus like particle either directly or indirectly (e.g. via a linker group). In some embodiments, the MTO or an analog thereof is directly attached to a functional group capable of reacting with the agent. For example, a nucleophilic group, such as an amino or sulfhydryl group, can be capable of reacting with a carbonyl-containing group, such as an anhydride or an acid halide, or with an alkyl group containing a good leaving group (e.g., a halide). Alternatively, a suitable chemical linker group can be used. A linker group can serve to increase the chemical reactivity of a substituent on either the agent or the virus particle, and thus increase the coupling efficiency. A preferred group suitable as a site for attaching MTO or an analogs thereof to the virus particle is one or more lysine residues present in the viral coat protein that have a free amino group that can be capable of reacting with a carbonyl-containing group, such as an anhydride or an acid halide, or with an alkyl group containing a good leaving group (e.g., a halide). Viral coat proteins also contain glutamic and aspartic acids. The carboxylate groups of these amino acids also present attractive targets for functionalization using carbodiimide activated linker molecules; cysteines can also be present which facilitate chemical coupling via thiol-selective chemistry (e.g., maleimide-activated compounds). Further, viral coat proteins contain tyrosines, which can be modified using diazonium coupling reactions. In addition, genetic modification can be applied to introduce any desired functional residue, including non-natural amino acids, e.g. alkyne- or azide-functional groups. See Hermanson, G. T. Bioconjugation Techniques. (Academic Press, 2008) and Pokorski, J. K. and N. F. Steinmetz, Mol Pharm 8(1): 29-43 (2011), the disclosures of which are incorporated herein by reference.

Alternatively, a suitable chemical linker group can be used. A linker group can serve to increase the chemical reactivity of a substituent on either the agent or the rod-shaped virus particle or virus-like particle, and thus increase the coupling efficiency. Suitable linkage chemistries include maleimidyl linkers, which can be used to link to thiol groups, isothiocyanate and succinimidyl (e.g., N-hydroxysuccinimidyl (NHS)) linkers, which can link to free amine groups, diazonium which can be used to link to phenol, and amines, which can be used to link with free acids such as carboxylate groups using carbodiimide activation. Useful functional groups are present on viral coat proteins based on the particular amino acids present, and additional groups can be designed into recombinant viral coat proteins. It will be evident to those skilled in the art that a variety of bifunctional or polyfunctional reagents, both homo- and hetero-functional (such as those described in the catalog of the Pierce Chemical Co., Rockford, Ill.), can be employed as a linker group. Coupling can be effected, for example, through amino groups, carboxyl groups, sulfhydryl groups or oxidized carbohydrate residues.

Other types of linking chemistries are also available. For example, methods for conjugating polysaccharides to peptides are exemplified by, but not limited to coupling via alpha- or epsilon-amino groups to NaIO₄-activated oligosaccharide (Bocher et al., J. Immunol. Methods 27, 191-202 (1997)), using squaric acid diester (1,2-diethoxycyclobutene-3,4-dione) as a coupling reagent (Tietze et al. Bioconjug Chem. 2:148-153 (1991)), coupling via a peptide linker wherein the polysaccharide has a reducing terminal and is free of carboxyl groups (U.S. Pat. No. 5,342,770), and coupling with a synthetic peptide carrier derived from human heat shock protein hsp65 (U.S. Pat. No. 5,736,146). Further methods for conjugating polysaccharides, proteins, and lipids to plant virus peptides are described by U.S. Pat. No. 7,666,624.

Some embodiments described herein also relate to methods of treating cancer in a subject in need thereof by administering to the subject a therapeutically effective amount of an anti-cancer virus particle, the virus particle including a rod-shaped plant virus or virus-like particle and mitoxantrone (MTO) or analogs thereof, wherein the MTO is loaded into the interior channel of the rod-shaped plant virus particle.

“Cancer” or “malignancy” are used as synonymous terms and refer to any of a number of diseases that are characterized by uncontrolled, abnormal proliferation of cells, the ability of affected cells to spread locally or through the bloodstream and lymphatic system to other parts of the body (i.e., metastasize) as well as any of a number of characteristic structural and/or molecular features. A “cancer cell” refers to a cell undergoing early, intermediate or advanced stages of multi-step neoplastic progression. The features of early, intermediate and advanced stages of neoplastic progression have been described using microscopy. Cancer cells at each of the three stages of neoplastic progression generally have abnormal karyotypes, including translocations, inversion, deletions, isochromosomes, monosomies, and extra chromosomes. Cancer cells include “hyperplastic cells,” that is, cells in the early stages of malignant progression, “dysplastic cells,” that is, cells in the intermediate stages of neoplastic progression, and “neoplastic cells,” that is, cells in the advanced stages of neoplastic progression.

The cancers treated by a method described herein can include the following: leukemias, such as but not limited to, acute leukemia, acute lymphocytic leukemia, acute myelocytic leukemias, such as, myeloblastic, promyelocytic, myelomonocytic, monocytic, and erythroleukemia leukemias and myelodysplastic syndrome; chronic leukemias, such as but not limited to, chronic myelocytic (granulocytic) leukemia, chronic lymphocytic leukemia, hairy cell leukemia; polycythemia vera; lymphomas such as but not limited to Hodgkin's disease, non-Hodgkin's disease; multiple myelomas such as but not limited to smoldering multiple myeloma, nonsecretory myeloma, osteosclerotic myeloma, plasma cell leukemia, solitary plasmacytoma and extramedullary plasmacytoma; Waldenstrom's macroglobulinemia; monoclonal gammopathy of undetermined significance; benign monoclonal gammopathy; heavy chain disease; bone and connective tissue sarcomas such as but not limited to bone sarcoma, osteosarcoma, chondrosarcoma, Ewing's sarcoma, malignant giant cell tumor, fibrosarcoma of bone, chordoma, periosteal sarcoma, soft-tissue sarcomas, angiosarcoma (hemangiosarcoma), fibrosarcoma, Kaposi's sarcoma, leiomyosarcoma, liposarcoma, lymphangiosarcoma, neurilemmoma, rhabdomyosarcoma, synovial sarcoma; brain tumors such as but not limited to, glioma, astrocytoma, glioblastoma, brain stem glioma, ependymoma, oligodendroglioma, nonglial tumor, acoustic neurinoma, craniopharyngioma, medulloblastoma, meningioma, pineocytoma, pineoblastoma, primary brain lymphoma; breast cancer including but not limited to ductal carcinoma, adenocarcinoma, lobular (small cell) carcinoma, intraductal carcinoma, medullary breast cancer, mucinous breast cancer, tubular breast cancer, papillary breast cancer, Paget's disease, and inflammatory breast cancer; adrenal cancer such as but not limited to pheochromocytoma and adrenocortical carcinoma; thyroid cancer such as but not limited to papillary or follicular thyroid cancer, medullary thyroid cancer and anaplastic thyroid cancer; pancreatic cancer such as but not limited to, insulinoma, gastrinoma, glucagonoma, vipoma, somatostatin-secreting tumor, and carcinoid or islet cell tumor; pituitary cancers such as but limited to Cushing's disease, prolactin-secreting tumor, acromegaly, and diabetes insipius; eye cancers such as but not limited to ocular melanoma such as iris melanoma, choroidal melanoma, and cilliary body melanoma, and retinoblastoma; vaginal cancers such as squamous cell carcinoma, adenocarcinoma, and melanoma; vulvar cancer such as squamous cell carcinoma, melanoma, adenocarcinoma, basal cell carcinoma, sarcoma, and Paget's disease; cervical cancers such as but not limited to, squamous cell carcinoma, and adenocarcinoma; uterine cancers such as but not limited to endometrial carcinoma and uterine sarcoma; ovarian cancers such as but not limited to, ovarian epithelial carcinoma, borderline tumor, germ cell tumor, fallopian tube cancer, and stromal tumor; esophageal cancers such as but not limited to, squamous cancer, adenocarcinoma, adenoid cystic carcinoma, mucoepidermoid carcinoma, adenosquamous carcinoma, sarcoma, melanoma, plasmacytoma, verrucous carcinoma, and oat cell (small cell) carcinoma; stomach cancers such as but not limited to, adenocarcinoma, fungating (polypoid), ulcerating, superficial spreading, diffusely spreading, malignant lymphoma, liposarcoma, fibrosarcoma, and carcinosarcoma; colon cancers; rectal cancers; liver cancers such as but not limited to hepatocellular carcinoma and hepatoblastoma; gallbladder cancers such as adenocarcinoma; cholangiocarcinomas such as but not limited to papillary, nodular, and diffuse; lung cancers such as non-small cell lung cancer, squamous cell carcinoma (epidermoid carcinoma), adenocarcinoma, large-cell carcinoma and small-cell lung cancer; testicular cancers such as but not limited to germinal tumor, seminoma, anaplastic, classic (typical), spermatocytic, nonseminoma, embryonal carcinoma, teratoma carcinoma, choriocarcinoma (yolk-sac tumor), prostate cancers such as but not limited to, prostatic intraepithelial neoplasia, adenocarcinoma, leiomyosarcoma, and rhabdomyosarcoma; penal cancers; oral cancers such as but not limited to squamous cell carcinoma; basal cancers; salivary gland cancers such as but not limited to adenocarcinoma, mucoepidermoid carcinoma, and adenoidcystic carcinoma; pharynx cancers such as but not limited to squamous cell cancer, and verrucous; skin cancers such as but not limited to, basal cell carcinoma, squamous cell carcinoma and melanoma, superficial spreading melanoma, nodular melanoma, lentigo malignant melanoma, acral lentiginous melanoma; kidney cancers such as but not limited to renal cell carcinoma, adenocarcinoma, hypemephroma, fibrosarcoma, transitional cell cancer (renal pelvis and/or uterer); Wilms' tumor; bladder cancers such as but not limited to transitional cell carcinoma, squamous cell cancer, adenocarcinoma, carcinosarcoma. In addition, cancers include myxosarcoma, osteogenic sarcoma, endotheliosarcoma, lymphangioendotheliosarcoma, mesothelioma, synovioma, hemangioblastoma, epithelial carcinoma, cystadenocarcinoma, bronchogenic carcinoma, sweat gland carcinoma, sebaceous gland carcinoma, papillary carcinoma and papillary adenocarcinomas (for a review of such disorders, see Fishman et al., 1985, Medicine, 2d Ed., J. B. Lippincott Co., Philadelphia and Murphy et al., 1997, Informed Decisions: The Complete Book of Cancer Diagnosis, Treatment, and Recovery, Viking Penguin, Penguin Books U.S.A., Inc., United States of America).

In some embodiments, the cancer is selected from the group consisting of breast cancer, prostate cancer, fibrosarcoma, leukemia and lymphoma. In some embodiments, the cancer is metastatic breast cancer. In some embodiments, the breast cancer is triple negative breast cancer.

In some embodiments, the subject being administered a therapeutically effective amount of an anti-cancer plant virus particle is a subject who has been identified as having cancer. As is known to those skilled in the art, there are a variety of methods of identifying (i.e., diagnosing) a subject who has cancer. For example, diagnosis of cancer can include one or more of a physical exam, laboratory tests, imaging analysis, and biopsy. After cancer is diagnosed, a variety of tests may be carried out to look for specific features characteristic of different types and or the extent of cancer in the subject. These tests include, but are not limited to, bone scans, X-rays, immunophenotyping, flow cytometry, and fluorescence in situ hybridization testing. Typical methods of diagnosing triple-negative breast cancer can include, but are not limited to, a physical exam, digital mammogram, breast MRI, breast ultrasound, stereotactic core and/or open tumor biopsy, as well as lab tests to determine if the tumor tissue expresses estrogen, progesterone, and HER-2/neu.

In some embodiments, the rod-shaped plant virus or VLP is used to target cancer cells or cancer tissue in a subject. As used herein, targeting cancer tissue includes the ability of the anti-cancer virus particles to reach and preferably accumulate at site of cancer after being administered to the subject, for example, where the anti-cancer virus particles are systemically administered to a subject. The ability of rod-shaped plant virus particles to target cancer tissue is supported by the in vitro cell uptake and animal model in vivo drug delivery studies carried out by the inventors. See International Patent Publication WO/2013/181557, the disclosure of which is incorporated herein by reference. While not intending to be bound by theory, it appears that rod-shaped plant virus particles are drawn to the leaky vasculature caused by the angiogenesis associated with rapid tumor growth, and this leaky vasculature encourages entry for anti-cancer plant virus particles through small pores, thereby delivering the anti-cancer plant virus particles to the cancer cells. As a result of this preferential accumulation, embodiments of the invention can deliver about 10%, about 20%, about 30%, about 40%, or even about 50% or more of the injected dose to tumor tissue.

In some embodiments, the administration of the virus particle can be proximal to a tumor in the subject or directly to the tumor site to provide a high local concentration of the rod-shaped virus particle or virus-like particle loaded with MTO or an analog thereof in the tumor microenvironment (TME). In certain embodiments, release of the MTO or an analog thereof from the rod-shaped plant virus particle or VLP is pH dependent and release is triggered by an acidic environments, such as the tumor microenvironment.

In some embodiments, a targeting moiety can also be attached to the rod-shaped plant virus particle. By “targeting moiety” herein is meant a functional group which serves to target or direct the virus particle to a particular location, cell type, diseased tissue, or association. In general, the targeting moiety is directed against a target molecule. Thus, for example, antibodies, cell surface receptor ligands and hormones, lipids, sugars and dextrans, alcohols, bile acids, fatty acids, amino acids, peptides and nucleic acids may all be attached to localize or target the anti-lymphoma plant virus particle to a particular site. In some embodiments, the targeting moiety allows targeting of the plant virus particles of the invention to a particular tissue or the surface of a cell. Preferably, the targeting moiety is linked to the exterior surface of the rod-shaped virus particle or VLP to provide easier access to the target molecule.

In some embodiments, the targeting moiety is a peptide. In further embodiments, the targeting moiety is an antibody. The term “antibody” includes antibody fragments, as are known in the art, including Fab Fab₂, single chain antibodies (Fv for example), chimeric antibodies, etc., either produced by the modification of whole antibodies or those synthesized de novo using recombinant DNA technologies. In further embodiments, the antibody targeting moieties of the invention are humanized antibodies or human antibodies. Humanized forms of non-human (e.g., murine) antibodies are chimeric immunoglobulins, immunoglobulin chains or fragments thereof (such as Fv, Fab, Fab′, F(ab′)₂ or other antigen-binding subsequences of antibodies) which contain minimal sequence derived from non-human immunoglobulin.

In some embodiments, the antibody is directed against a cell-surface marker on a cancer cell; that is, the target molecule is a cell surface molecule. As is known in the art, there are a wide variety of cell surface molecules known to be differentially expressed on tumor cells, including, but not limited to, HER2. Examples of physiologically relevant carbohydrates may be used as cell-surface markers include, but are not limited to, antibodies against markers for breast cancer (CA 15-3, CA 549, CA 27.29), mucin-like carcinoma associated antigen (MCA), ovarian cancer (CA125), pancreatic cancer (DE-PAN-2), and colorectal and pancreatic cancer (CA 19, CA 50, CA242). In some embodiments, a cell surface molecule known to be differentially expressed on lymphoma cells is used. Examples of such cell surface markers include CD20, CD22, and CD40.

In some embodiments, a coating can be added to the exterior of the rod-shaped plant virus particle or VLP to improve bioavailability. Administering plant virus particles to a subject can sometimes generate an immune response. An “immune response” refers to the concerted action of lymphocytes, antigen presenting cells, phagocytic cells, granulocytes, and soluble macromolecules produced by the above cells or the liver (including antibodies, cytokines, and complement) that results in selective damage to, destruction of, or elimination from the human body of cancerous cells, metastatic tumor cells, invading pathogens, cells or tissues infected with pathogens, or, in cases of autoimmunity or pathological inflammation, normal human cells or tissues. Components of an immune response can be detected in vitro by various methods that are well known to those of ordinary skill in the art.

Generation of an immune response by the anti-cancer plant virus particles is typically undesirable. Accordingly, in some embodiments it may be preferable to modify the exterior of the plant virus particle or take other steps to decrease the immune response. For example, an immunosuppressant compound can be administered to decrease the immune response. More preferably, the anti-cancer plant virus particle can be modified to decrease its immunogenicity. Examples of methods suitable for decreasing immunity include attachment of anti-fouling (e.g., zwitterionic) polymers, glycosylation of the virus carrier, and PEGylation.

In some embodiments, the immunogenicity of virus particle is decreased by PEGylation. PEGylation is the process of covalent attachment of polyethylene glycol (PEG) polymer chains to a molecule such as a filamentous plant virus carrier. PEGylation can be achieved by incubation of a reactive derivative of PEG with the plant virus particle exterior. The covalent attachment of PEG to the anti-cancer plant virus particle can “mask” the agent from the host's immune system, and reduce production of antibodies against the carrier. PEGylation also may provide other benefits. PEGylation can be used to vary the circulation time of the filamentous plant virus carrier. For example, use of PEG 5,000 can provide an anti-lymphoma virus particle with a circulation half-life of about 12.5 minutes, while use of PEG 20,000 can provide an anti-cancer plant virus particle with a circulation half life of about 110 minutes.

The first step of PEGylation is providing suitable functionalization of the PEG polymer at one or both terminal positions of the polymer. The chemically active or activated derivatives of the PEG polymer are prepared to attach the PEG to the anti-lymphoma virus particle. There are generally two methods that can be used to carry out PEGylation; a solution phase batch process and an on-column fed-batch process. The simple and commonly adopted batch process involves the mixing of reagents together in a suitable buffer solution, preferably at a temperature between 4 and 6° C., followed by the separation and purification of the desired product using a chromatographic technique.

In some embodiments, a method of treating cancer described herein can further include can include administering an additional therapeutic or cancer therapy to the subject. A “cancer therapeutic” or “cancer therapy”, as used herein, can include any agent or treatment regimen that is capable of negatively affecting cancer in an animal, for example, by killing cancer cells, inducing apoptosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, or increasing the lifespan of an animal with cancer. Cancer therapeutics can include one or more therapies such as, but not limited to, chemotherapies, radiation therapies, hormonal therapies, and/or biological therapies/immunotherapies. A reduction, for example, in cancer volume, growth, migration, and/or dispersal in a subject may be indicative of the efficacy of a given therapy.

In some embodiments, the method can include the step of administering a therapeutically effective amount of an additional anticancer therapeutic agent to the subject. Additional anticancer therapeutic agents can be in the form of biologically active ligands, small molecules, peptides, polypeptides, proteins, DNA fragments, DNA plasmids, interfering RNA molecules, such as siRNAs, oligonucleotides, and DNA encoding for shRNA. In some embodiments, cytotoxic compounds are included in an anticancer agent described herein. Cytotoxic compounds include small-molecule drugs such as doxorubicin, methotrexate, vincristine, and pyrimidine and purine analogs, referred to herein as antitumor agents. In particular embodiments, an additional anticancer therapeutic agent can include a corticosteroid such as but not limited to prednisone.

The additional anticancer therapeutic agent can include an anticancer or an antiproliferative agent that exerts an antineoplastic, chemotherapeutic, antiviral, antimitotic, antitumorgenic, and/or immunotherapeutic effects, e.g., prevent the development, maturation, or spread of neoplastic cells, directly on the tumor cell, e.g., by cytostatic or cytocidal effects, and not indirectly through mechanisms such as biological response modification. There are large numbers of anti-proliferative agent agents available in commercial use, in clinical evaluation and in pre-clinical development. For convenience of discussion, anti-proliferative agents are classified into the following classes, subtypes and species: ACE inhibitors, alkylating agents, angiogenesis inhibitors, angiostatin, anthracyclines/DNA intercalators, anti-cancer antibiotics or antibiotic-type agents, antimetabolites, antimetastatic compounds, asparaginases, bisphosphonates, cGMP phosphodiesterase inhibitors, calcium carbonate, cyclooxygenase-2 inhibitors, DHA derivatives, DNA topoisomerase, endostatin, epipodophylotoxins, genistein, hormonal anticancer agents, hydrophilic bile acids (URSO), immunomodulators or immunological agents, integrin antagonists, interferon antagonists or agents, MMP inhibitors, miscellaneous antineoplastic agents, monoclonal antibodies, nitrosoureas, NSAIDs, ornithine decarboxylase inhibitors, pBATTs, radio/chemo sensitizers/protectors, retinoids, selective inhibitors of proliferation and migration of endothelial cells, selenium, stromelysin inhibitors, taxanes, vaccines, and vinca alkaloids.

The major categories that some anti-proliferative agents fall into include antimetabolite agents, alkylating agents, antibiotic-type agents, hormonal anticancer agents, immunological agents, interferon-type agents, and a category of miscellaneous antineoplastic agents. Some anti-proliferative agents operate through multiple or unknown mechanisms and can thus be classified into more than one category.

Examples of anticancer therapeutic agents that can be administered in combination with a plant virus or virus-like particle described herein include Taxol, Adriamycin, dactinomycin, bleomycin, vinblastine, cisplatin, acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cladribine; crisnatol mesylate; cyclophosphamide; cytarabine; dacarbazine; daunorubicin hydrochloride; decitabine; dexormaplatin; dezaguanine; dezaguanine mesylate; diaziquone; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; ilmofosine; interleukin II (including recombinant interleukin II, or rIL2), interferon alfa-2a; interferon alfa-2b; interferon alfa-n1; interferon alfa-n3; interferon beta-I a; interferon gamma-I b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; temozolomide, teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride.

In certain embodiments, additional therapeutic agents administered to a subject for the treatment of triple negative breast cancer as described herein can include one or more of an anthracycline, such as adriamycin, an alkylating agent such as Cytoxan (cyclophosphamide), an antimetabolite such as Fluorouracil (5FU), and a taxane, such as Taxol or Taxotere.

In some embodiments, the anti-cancer therapy administered to the subject in addition to the anti-cancer plant virus particles can include the cancer ablation therapy. Ablating the cancer can be accomplished using a method selected from the group consisting of cryoablation, thermal ablation, radiotherapy, chemotherapy, radiofrequency ablation, electroporation, alcohol ablation, high intensity focused ultrasound, photodynamic therapy, administration of monoclonal antibodies, immunotherapy, and administration of immunotoxins.

In some embodiments, ablating the cancer includes immunotherapy of the cancer. Cancer immunotherapy is based on therapeutic interventions that aim to utilize the immune system to combat malignant diseases. It can be divided into unspecific approaches and specific approaches. Unspecific cancer immunotherapy aims at activating parts of the immune system generally, such as treatment with specific cytokines known to be effective in cancer immunotherapy (e.g., IL-2, interferon's, cytokine inducers). In contrast, specific cancer immunotherapy is based on certain antigens that are preferentially or solely expressed on cancer cells or predominantly expressed by other cells in the context of malignant disease (usually in vicinity of the tumor site). Specific cancer immunotherapy can be grouped into passive and active approaches.

In passive specific cancer immunotherapy substances with specificity for certain structures related to cancer that are derived from components of the immune system are administered to the patient. The most prominent and successful approaches are treatments with humanized or mouse/human chimeric monoclonal antibodies against defined cancer associated structures (such as Trastuzumab, Rituximab, Cetuximab, Bevacizumab, Alemtuzumab). The pharmacologically active substance exerts is activity as long as a sufficient concentration is present in the body of the patient, therefore administrations have to be repeated based on pharmacokinetic and pharmacodynamic considerations.

On the other hand, active specific cancer immunotherapy aims at antigen-specific stimulation of the patient's immune system to recognize and destroy cancer cells. Active specific cancer immunotherapy therefore, in general, is a therapeutic vaccination approach. There are many types of cancer vaccine approaches being pursued, such as vaccination with autologous or allogeneic whole tumor cells (in most cases genetically modified for better immune recognition), tumor cell lysates, whole tumor associated antigens (produced by means of genetic engineering or by chemical synthesis), peptides derived from protein antigens, DNA vaccines encoding for tumor associated antigens, surrogates of tumor antigens such as anti-idiotypic antibodies used as vaccine antigens, and the like. These manifold approaches are usually administered together with appropriate vaccine adjuvants and other immunomodulators in order to elicit a quantitatively and qualitatively sufficient immune response (many novel vaccine adjuvant approaches are being pursued in parallel with the development of cancer vaccines). Another set of cancer vaccine approaches relies on manipulating dendritic cells (DC) as the most important antigen presenting cell of the immune system. For example, loading with tumor antigens or tumor cell lysates, transfection with genes encoding for tumor antigens and in-vivo targeting are suitable immunotherapies that can be used together with the virus or virus-like particles of the invention for cancer treatment.

In some embodiments, ablating the cancer includes administering a therapeutically effective amount of radiotherapy (RT) to the subject. In some embodiments, RT is administered prior to administration of the rod-shaped plant virus nanoparticle. In some embodiments, administering to the cancer, (e.g., at a tumor site) a therapeutically effective amount of a rod-shaped plant virus or virus-like particle loaded with MTO to the subject in combination with administering radiotherapy to the subject can result in an increase in tumor infiltrating lymphocytes (TILs), such as tumor infiltrating neutrophils (TINs) at the tumor site of the subject.

Radiotherapy uses high-energy rays to treat disease, usually x-rays and similar rays (such as electrons). Radiotherapy administered to a subject can include both external and internal. External radiotherapy (or external beam radiation) aims high-energy x-rays at the tumor site including in some cases the peri-tumor margin. External radiotherapy typically includes the use of a linear accelerator (e.g., a Varian 2100C linear accelerator). External radiation therapy can include three-dimensional conformal radiation therapy (3D-CRT), image guided radiation therapy (IGRT), intensity modulated radiation therapy (IMRT), helical-tomotherapy, photon beam radiation therapy, proton beam radiation therapy, stereotactic radiosurgery and/or sterotactic body radiation therapy (SBRT).

Internal radiotherapy (brachytherapy) involves having radioactive material placed inside the body and allows a higher dose of radiation in a smaller area than might be possible with external radiation treatment. It uses a radiation source that is usually sealed in an implant. Exemplary implants include pellets, seeds, ribbons, wires, needles, capsules, balloons, or tubes. Implants are placed in your body, very close to or inside the tumor. Internal radiotherapy can include intracavitary or interstitial radiation. During intracavitary radiation, the radioactive source is placed in a body cavity (space), such as the uterus. With interstitial radiation, the implants are placed in or near the tumor, but not in a body cavity.

In some embodiments, a checkpoint inhibitor can be further administered to eradicate suppressive regulatory T cells prior to RT. Exemplary checkpoint inhibitors can include CTLA4 and PD-1/PDL-1 inhibitors. The cytotoxic T-lymphocyte-associated antigen 4 (CTLA-4) and programmed death 1 (PD-1) immune checkpoints are negative regulators of T-cell immune function and inhibition of these targets, results in increased activation of the immune system. Therefore, in some embodiments, a checkpoint inhibitor administered to a subject can include a CTLA-4 and/or PD-1 inhibitor. For example, Ipilimumab, an inhibitor of CTLA-4, is approved for the treatment of advanced or unresectable melanoma. Nivolumab and pembrolizumab, both PD-1 inhibitors, are approved to treat patients with advanced or metastatic melanoma and patients with metastatic, refractory non-small cell lung cancer. In addition, the combination of ipilimumab and nivolumab has been approved in patients with BRAF WT metastatic or unresectable melanoma.

It has been shown that moderate magnetic nanoparticle hyperthermia (mNPH) treatment administered to a tumor can generate immune-based systemic resistance to tumor rechallenge. Therefore, in some embodiments, a therapeutically effective amount of a moderate magnetic nanoparticle hyperthermia (mNPH) treatment can be administered to the subject in combination with an anti-cancer plant virus particle or virus-like particle and/or radiotherapy, wherein the mNPH is activated with an alternating magnetic field (AMF) to produce moderate heat. Without being bound by theory, it is believed that plant virus-like particle immune adjuvants, such as a plant virus nanoparticle and/or a mNPH, will combine with RT-induced generation of immunogenic cell death (ICD) to expand the tumor specific effector T cell population causing longer local and distant tumor remission.

A mNPH treatment can include the use of a magnetic iron oxide nanoparticle (IONP). Once administered to the subject intratumorally, the mNPH can, in some embodiments, be activated with an alternating magnetic field (AMF) to produce moderate heat (e.g., 43°/60° min) at the tumor site. In some embodiments, the RT is hypofractionated RT (HFRT) that delivers larger but fewer doses/fractions than typical RT therapies.

When used in vivo, the anti-cancer plant virus particles and/or additional anti-cancer therapeutic agents described herein can be administered as a pharmaceutical composition, comprising a mixture, and a pharmaceutically acceptable carrier. The anti-cancer virus particles may be present in a pharmaceutical composition in an amount from 0.001 to 99.9 wt %, more preferably from about 0.01 to 99 wt %, and even more preferably from 0.1 to 95 wt %.

The anti-cancer plant virus particles, or pharmaceutical compositions comprising these particles, may be administered by any method designed to provide the desired effect. Administration may occur enterally or parenterally; for example orally, rectally, intracisternally, intravaginally, intraperitoneally or locally. Parenteral administration methods include intravascular administration (e.g., intravenous bolus injection, intravenous infusion, intra-arterial bolus injection, intra-arterial infusion and catheter instillation into the vasculature), peri- and intra-target tissue injection, subcutaneous injection or deposition including subcutaneous infusion (such as by osmotic pumps), intramuscular injection, intraperitoneal injection, intracranial and intrathecal administration for CNS tumors, and direct application to the target area, for example by a catheter or other placement device.

For parenteral administration, compositions of the invention can be administered as injectable dosages of a solution or suspension of the substance in a physiologically acceptable diluent with a pharmaceutical carrier that can be a sterile liquid such as water oils, saline, glycerol, or ethanol. Additionally, auxiliary substances, such as wetting or emulsifying agents, surfactants, pH buffering substances and the like can be present in compositions. Other components of pharmaceutical compositions are those of petroleum, animal, vegetable, or synthetic origin, for example, peanut oil, soybean oil, and mineral oil. In general, glycols such as propylene glycol or polyethylene glycol are preferred liquid carriers, particularly for injectable solutions.

The pharmaceutical compositions can also include, depending on the formulation desired, pharmaceutically-acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, physiological phosphate-buffered saline, Ringer's solutions, dextrose solution, and Hank's solution. In addition, the pharmaceutical composition or formulation may also include other carriers, adjuvants, or nontoxic, nontherapeutic, nonimmunogenic stabilizers and the like.

Suitable pharmaceutically acceptable carriers may contain inert ingredients which do not unduly inhibit the biological activity of the compounds. The pharmaceutically acceptable carriers should be biocompatible, e.g., non-toxic, non-inflammatory, non-immunogenic and devoid of other undesired reactions upon the administration to a subject. Standard pharmaceutical formulation techniques can be employed, such as those described in Remington's Pharmaceutical Sciences, ibid. Suitable pharmaceutical carriers for parenteral administration include, for example, sterile water, physiological saline, bacteriostatic saline (saline containing about 0.9% mg/ml benzyl alcohol), phosphate-buffered saline, Hank's solution, Ringer's-lactate and the like. Methods for encapsulating compositions (such as in a coating of hard gelatin or cyclodextran) are known in the art (Baker, et al., “Controlled Release of Biological Active Agents”, John Wiley and Sons, 1986).

A pharmaceutically acceptable carrier for a pharmaceutical composition can also include delivery systems known to the art for entraining or encapsulating drugs, such as anticancer drugs. In some embodiments, the disclosed compounds can be employed with such delivery systems including, for example, liposomes, nanoparticles, nanospheres, nanodiscs, dendrimers, and the like. See, for example Farokhzad, O. C., Jon, S., Khademhosseini, A., Tran, T. N., Lavan, D. A., and Langer, R. (2004). “Nanoparticle-aptamer bioconjugates: a new approach for targeting prostate cancer cells.” Cancer Res., 64, 7668-72; Dass, C. R. (2002). “Vehicles for oligonucleotide delivery to tumours.” J. Pharm. Pharmacol., 54, 3-27; Lysik, M. A., and Wu-Pong, S. (2003). “Innovations in oligonucleotide drug delivery.” J. Pharm. Sci., 92, 1559-73; Shoji, Y., and Nakashima, H. (2004). “Current status of delivery systems to improve target efficacy of oligonucleotides.” Curr. Pharm. Des., 10, 785-96; Allen, T. M., and Cullis, P. R. (2004). “Drug delivery systems: entering the mainstream.” Science, 303, 1818-22. The entire teachings of each reference cited in this paragraph are incorporated herein by reference.

Suitable doses can vary widely depending on the therapeutic being used. A typical pharmaceutical composition for intravenous administration would be about 0.1 mg to about 10 g per subject per day. However, in other embodiments, doses from about 1 mg to about 1 g, or from about 10 mg to about 1 g can be used. Single or multiple administrations of the compositions may be administered depending on the dosage and frequency as required and tolerated by the subject. In any event, the administration regime should provide a sufficient quantity of the composition of this invention to effectively treat the subject.

Useful dosages of the additional anticancer agents, such as antimitotic agents, and anti-cancer plant virus particles can be determined by comparing their in vitro activity and the in vivo activity in animal models. Methods for extrapolation of effective dosages in mice, and other animals, to humans are known in the art; for example, see U.S. Pat. No. 4,938,949. An amount adequate to accomplish therapeutic or prophylactic treatment is defined as a therapeutically- or prophylactically-effective dose. In both prophylactic and therapeutic regimes, agents are usually administered in several dosages until an effect has been achieved. Effective doses of the additional anticancer agents and/or anti-cancer plant virus particles vary depending upon many different factors, including means of administration, target site, physiological state of the patient, whether the patient is human or an animal, other medications administered, and whether treatment is prophylactic or therapeutic.

The formulations may be conveniently presented in unit dosage form and may be prepared by any of the methods well known in the art of pharmacy. Preferably, such methods include the step of bringing the virus particles into association with a pharmaceutically acceptable carrier that constitutes one or more accessory ingredients. In general, the formulations are prepared by uniformly and intimately bringing the active agent into association with a liquid carrier, a finely divided solid carrier, or both, and then, if necessary, shaping the product into the desired formulations. The methods of the invention include administering to a subject, preferably a mammal, and more preferably a human, the composition of the invention in an amount effective to produce the desired effect.

One skilled in the art can readily determine an effective amount of anti-cancer plant virus particles and/or additional cancer therapeutics to be administered to a given subject, by taking into account factors such as the size and weight of the subject; the extent of disease penetration; the age, health and sex of the subject; the route of administration; and whether the administration is local or systemic. Those skilled in the art may derive appropriate dosages and schedules of administration to suit the specific circumstances and needs of the subject. For example, suitable doses of the anti-cancer virus particles to be administered can be estimated from the volume of cancer cells to be killed or volume of tumor to which the virus particles are being administered.

Useful dosages of the active agents can be determined by comparing their in vitro activity and the in vivo activity in animal models. Methods for extrapolation of effective dosages in mice, and other animals, to humans are known in the art. An amount adequate to accomplish therapeutic or prophylactic treatment is defined as a therapeutically- or prophylactically-effective dose. In both prophylactic and therapeutic regimes, agents are usually administered in several dosages until an effect has been achieved. Effective doses of the virus particles vary depending upon many different factors, including means of administration, target site, physiological state of the patient, whether the patient is human or an animal, characteristics of the subject, such as general health, age, sex, body weight and tolerance to drugs as well as the degree, severity and type of cancer, other medications administered, and whether treatment is prophylactic or therapeutic. The skilled artisan will be able to determine appropriate dosages depending on these and other factors using standard clinical techniques.

The methods described herein contemplate single as well as multiple administrations, given either simultaneously or over an extended period of time. A pharmaceutically acceptable composition containing the anti-cancer virus particles and/or additional cancer therapeutic can be administered at regular intervals, depending on the nature and extent of the cancer's effects, and on an ongoing basis. Administration at a “regular interval,” as used herein, indicates that the therapeutically effective amount is administered periodically (as distinguished from a one-time dose). In one embodiment, the pharmaceutically acceptable composition containing the anti-cancer plant virus particles and/or an additional cancer therapeutic is administered periodically, e.g., at a regular interval (e.g., bimonthly, monthly, biweekly, weekly, twice weekly, daily, twice a day or three times or more often a day).

The administration interval for a single individual can be fixed, or can be varied over time, depending on the needs of the individual. For example, in times of physical illness or stress, or if disease symptoms worsen, the interval between doses can be decreased.

For example, the administration of anti-cancer virus particles and/or the additional therapeutic agent can take place at least once on day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or alternatively, at least once on week 1, 2, 3, 4, 5, 6, 7, 8, 9, 10, 11, 12, 13, 14, 15, 16, 17, 18, 19 or 20, or any combination thereof, using single or divided doses of every 60, 48, 36, 24, 12, 8, 6, 4, or 2 hours, or any combination thereof. Administration can take place at any time of day, for example, in the morning, the afternoon or evening. For instance, the administration can take place in the morning, e.g., between 6:00 a.m. and 12:00 noon; in the afternoon, e.g., after noon and before 6:00 p.m.; or in the evening, e.g., between 6:01 p.m. and midnight.

In an exemplary embodiment, anti-cancer TMV plant virus particles are loaded with MTO and administered to the subject in need thereof via IV injection at 1 mg kg⁻¹ on days 1, 5, and 10.

In some embodiments, the frequency of administration of anti-cancer virus particles can pose challenging for clinical implementation. Therefore, in some embodiments, the anti-cancer virus particles administered to a subject can be formulated in a slow release formulation in order to sustain immune stimulation by maintaining a therapeutic concentration of the anti-cancer virus particles, (e.g., at the site of a tumor) while alleviating the need for frequent administrations. In some embodiments, a slow release formulation can include a polymer-based hydrogel or a dendrimer.

In some embodiments, a slow-release formulation can include an anti-cancer plant virus or virus like particle dendrimer hybrid aggregate. The dendrimer can include a positively-charged polyamidoamine (PAMAM) dendrimer, such as a medium-sized generation 3 (G3) or generation 4 (G4) PAMAM dendrimer. Depending on the specific application, the plant virus-like particle-dendrimer hybrid aggregates can vary in size and release rate of the plant virus-like particle from the dendrimer when administered to a subject. In some embodiments, the anti-cancer virus particle-dendrimer hybrid aggregates are formulated so that at low salt the assembly of the aggregates is triggered and while under physiologic salt concentrations disassembly and anti-cancer virus particle release is induced.

Examples have been included to more clearly describe particular embodiments of the invention. However, there are a wide variety of other embodiments within the scope of the present invention, which should not be limited to the particular examples provided herein.

### Example 1

Mitoxantrone (MTO) is a topoisomerase II inhibitor which has been used to treat forms of cancer either as solo chemotherapy regimen or as component in cocktail treatments. However, as with other anti-neoplastic agents, MTO has severe cardiac side effects. Therefore, a drug delivery approach holds promise to improve safety and applicability of this chemotherapy regimen. In this Example, we show the application of a plant virus-based nanotechnology derived from tobacco mosaic virus (TMV) as a delivery vehicle for MTO towards cancer therapy. TMV is a high aspect ratio soft matter nanotube with dimensions of 300×18 nm and 4-nm wide channel. The surface chemistry of the interior and exterior surfaces is distinct and we established charge-driven drug loading mechanisms to accommodate therapeutics inside the channel for drug delivery. We demonstrate effective MTO loading into TMV yielding ˜1,000 MTO per TMV carrier. Treatment efficacy of MTO-loaded TMV (MTO-TMV) was assessed in in-vitro and in-vivo models. In-vitro testing confirmed that MTO maintained its efficacy when delivered by TMV in a panel of cancer cell lines. Drug delivery in-vivo using a mouse model of triple negative breast cancer demonstrated superior efficacy of TMV-delivered MTO vs. free MTO. We hypothesized that TMV delivery of MTO may overcome cardiotoxicity while enhancing drug delivery and thus overall treatment efficacy.

### Methods

### Synthesis of MTO-TMV

T158K mutant of TMV (in the following referred to as TMV) was produced by mechanical inoculation of Nicotiana benthamiana plants and established purification protocols. 1 mg ml₋₁ of TMV was mixed in the dark with 10,000-fold molar excess of mitoxantrone (Sigma-Aldrich, MTO) in 10 mM potassium phosphate (KP) buffer at pH 7.4 for 18 hours. The reaction mix was purified by centrifugation at 112,000 g for 1 hour over a 40% (w/v) sucrose cushion to remove any excess of free MTO. The resulting MTO-TMV pellet was resuspended in 10 mM KP buffer pH 7.4 and further purified by centrifugation at 16,000 g for 10 minutes to remove any particle aggregates. Finally, the MTO-TMV were eluted through a GE Healthcare PD Minitrap G-25 column to remove any remaining free MTO. UV-visible spectroscopy (UV-vis) was used to determine the respective concentration of TMV and MTO.

Samples were also analyzed by transmission electron microscopy (TEM) and size exclusion chromatography (SEC) to confirm particle monodispersity and integrity.

### UV-Vis Spectroscopy

The UV-vis Spectra of TMV and _{MTO}TMV were analyzed with a NanoDrop spectrophotometer (Thermo Scientific). The molar ratio of MTO loading was determined by comparing the ratio of MTO:TMV coat protein concentration, which was determined by analyzing their respective absorbance and Beer-Lambert law. The extinction coefficients of TMV and MTO are as follows: TMV ε (260 nm)=3 mL·mg⁻¹cm1⁻¹, MTO ε (622 nm)=25,000 M⁻¹ cm⁻¹. The molecular weight of TMV and MTO are 39.4×10⁶ g mol⁻¹ and 514.71 g mol⁻¹ respectively. It is noted that because MTO also absorbs at 260 nm, the MTO contribution at 260 nm is therefore subtracted when determining the TMV concentration.

### Size Exclusion Chromatography (SEC) by Fast Protein Liquid Chromatography (FPLC)

Samples (200 μL at 1 mg ml⁻¹ of protein) were eluted through a Superose6 column on the ÄKTA Explorer chromatography system (GE Healthcare) using a flow rate of 0.5 mL/min in 10 mM KP pH 7.4. The absorbance at 260 (TMV RNA), 280 (TMV protein), and 622 nm (MTO) was recorded.

### Transmission Electron Microscopy (TEM)

A 20 μL drop of TMV or _{MTO}TMV at 1 mg ml⁻¹ protein concentration was added to Formvar carbon film coated copper TEM grids (FCF400-CU, Electron Microscopy Sciences) for 2 min at room temperature. After two washing steps with deionized water, the grids were stained twice with 2% (w/v) uranyl acetate in deionized water for 45 s. A Tecnai F30 transmission electron microscope was used to image the prepared samples at 300 kV.

MTO Drug Release from _{MTO}TMV

_{MTO}TMV formulations (500 μL, 1 mg ml⁻¹) were placed in Slide-A-Lyzer MINI dialysis units (69570, Fisher) and dialyzed against 2 l of 10 mM KP buffer pH 5.0 and pH 7.4 over 72 hours and at 4° C. or room temperature (RT). 10 μL samples were removed at t=12, 16, 24, 48, and 72 hours after the start of dialysis, and analyzed by UV-vis spectroscopy to quantify the percent of MTO released from _{MTO}TMV. Drug release was calculated by comparing the remaining drug in the particle solution to the initial drug concentration.

_{MTO}TMV vs. MTO Cell Uptake

Cell uptake of _{MTO}TMV vs. MTO was assessed using MDA-MB-231 cells (triple negative breast cancer), HT1080 cells (fibrosarcoma), and PC-3 cells (prostate cancer). MDA-MB-231 and HT1080 cells were cultured in high glucose Dulbecco's modified Eagle medium (DMEM) with 4 mM L-glutamine (Fisher). PC-3 cells were cultured in Rosewell Park Memorial Institute (RPMI) 1640 medium. All media were supplemented with 10% (v/v) FBS and 1% (v/v) penicillin-streptomycin. Cells were grown to confluency at 37° C. and 5% CO₂. Cells were seeded into an untreated V-bottomed 96-well plate at 250,000 cells per well in 200 μL of media. Triplicates of MTO or _{MTO}TMV were added at a concentration of 100,000 particles/cell and incubated for 16 hours at 37° C., 5% CO₂. Cells were then washed with 7.4 pH PBS containing 5% (v/v) FBS and 0.1% (w/v) sodium azide, and then fixed with 2% (v/v) paraformaldehyde 7.4 pH PBS for 15 minutes. Cells were sorted by fluorescence using a R660 filter (405 nm Em/660 nm Ex) and a Accuri C6 Flow Cytometer. All experiments were carried out at least twice and triplicate samples were analyzed using FlowJo software.

### MTO-TMV MTO Cytotoxicity

Cell toxicity was evaluated using the MTT assay (ATCC) and MDA-MB-231, HT1080, and PC-3 cell lines. Cells were exposed to free MTO, _{MTO}TMV, TMV, and PBS for 24 hours in culture medium at 37° C. and 5% CO₂; MTO concentrations ranged from 10 μM to 100 μM with increments of factor 10; TMV concentration were matched to _{MTO}TMV. The assay was performed as per manufacturer's recommendation; a BioTek Synergy HT multidetection microplate reader was used for read-out.

MTO-TMV Vs. MTO Therapy Using the MDA-MB-231 Mouse Model of Triple Negative Breast Cancer

All animal studies were performed according to Case Western Reserve University's IACUC-approved procedures. Female NCR nu/nu mice were injected subcutaneously into the right flank using 2×10⁶ MDA-MB-231 cells suspended in 100 μL of media and Matrigel (Corning) at a 1:1 ratio. Once established, tumors were monitored every other day, with total tumor volume calculated using the formula v=l×w2, where l is the length and w the width of the tumor. Treatment injections were started when tumors reached a volume of 100 mm₃. Groups of n=5 animals were treated with MTO, _{MTO}TMV, TMV, and PBS. Treatment was delivered via IV injection at 1 mg kg₋₁ normalized to MTO on days 1, 5, and 10. Injection volumes did not exceed 300 μL.

### Results and Discussion

### _{MTO}TMV Synthesis and Characterization

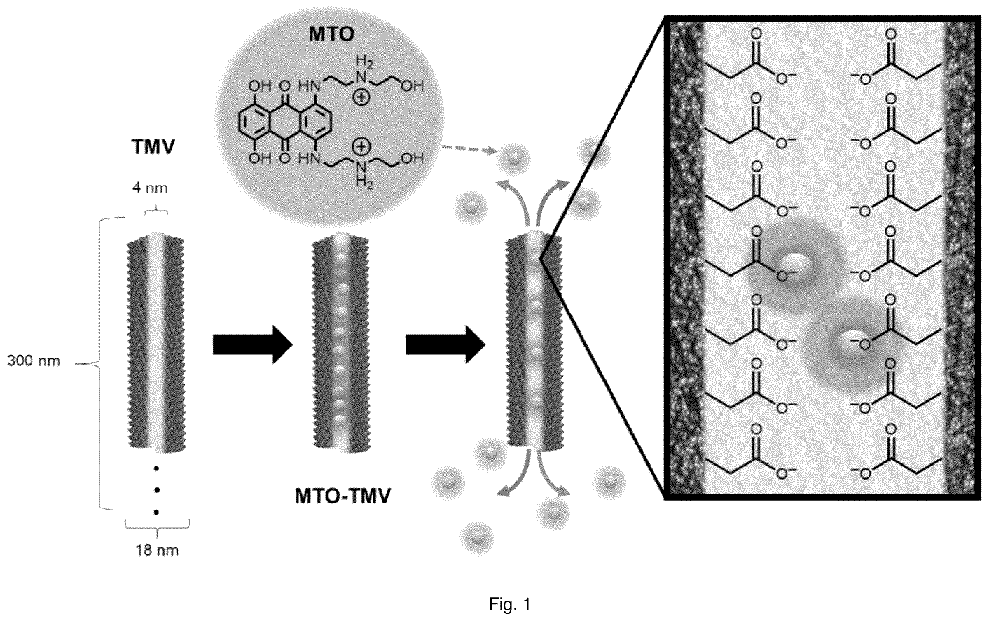

TMV was propagated in and purified from Nicotiana benthamiana plants at yields of up to 500 mg pure TMV per 100 gram of infected leaf tissue. TMV consists of 2,130 identical coat proteins (made of 158 amino acids) arranged in a helical structure around the single-stranded viral RNA. TMV virions form a cylindrical structure, its inner surface is lined with 4,260 solvent-exposed glutamic acid residues (Glu 97 and 106). These exposed carboxylic groups provide a negatively charged environment, which we have previously exploited for the encapsulation of positively charged therapeutics, such as chemotherapies, photosensitizers, and pesticides. MTO in its native state contains a +2 charge (FIG. 1) and measures approximately 1.4 nm across its longest axis; therefore, MTO has attributes making it an attractive candidate to be encapsulated into TMV via charge-driven interactions. To achieve this, MTO and TMV were mixed at a 10,000:1 MTO:TMV ratio overnight in 10 mM potassium phosphate (KP) buffer at pH 7.4. Following purification to remove any excess and MTO, the resulting MTO-loaded TMV particles, denoted MTOTMV, were analyzed to confirm their structural integrity and assess drug loading and release (FIG. 2).

Size exclusion chromatography (SEC) by fast liquid protein chromatography (FPLC) and transmission electron microscopy (TEM) confirmed that _{MTO}TMV particles maintained their structural integrity after MTO loading (FIG. 2A+C). TEM imaging shows high aspect ratio nanorods. It should be noted that while native TMV measures 300 nm, TEM imaging typically shows a distribution of particle lengths, which likely is an artifact from sample preparation leading to broken or fragmented particles; however, no differences were noted between TMV and _{MTO}TMV indicating stability of the MTOTMV formulation (FIG. 2C). The FPLC elution profile also shows the typical TMV profile with elution from the Superose6 column at ˜8 mLs; the ratio of 260:280 of 1.2 is indicative of intact TMV and overlap of the 622 nm peak with the 260/280 nm peaks indicates co-elution of MTO with TMV; free MTO was not detected in the preparation (FIG. 2A).

MTO drug loading into TMV was then quantified by UV-Vis spectroscopy (FIG. 2B) using Beer Lambert Law and the TMV and MTO-specific extinction coefficients; we determined the loading with approximately ˜1,000 MTO molecules per TMV particle. The degree of drug loading is comparable to other TMV-drug delivery systems that we previously described. For example, we reported similar degree of drug loading using a porphyrin-based photosensitizer. It should be noted that with platinum drugs, using either cisplatin or its monofunctional derivatives phenanthriplatin, loading with up to 2,000 drugs per TMV could be achieved. A recent structure-function study using a distinct set of phenanthriplatin analogs indicates that the net charge of phenanthriplatin analogs and their ionic mobilities have no effect on loading—however an increased number of heteroaromatic rings of the platinum ligand appears to enhance loading efficiency, possibly by stabilizing the hydrophobic interactions and stacking inside the TMV channel. MTO is known to interact and bind to proteins via hydrophobic interactions; therefore, we propose that MTO-TMV drug loading is driven by a combination of charge interactions and hydrophobic stacking. In fact, MTO may not be bound to the interior channel exclusively, but could also be bound to the exterior surface of the particles through non-specific drug-protein interactions.

Lastly, we assessed the release rate of MTO from TMV using dialysis against KP buffer pH 5.0 vs. 7.4 corresponding to the acidic tumor microenvironment and physiological conditions. We also considered testing at 4° C. and 22° C. to assess stability under storage conditions in the fridge or room temperature. While temperature only had a modest effect on drug release; faster drug release rates were observed under acidic conditions (t_{1/2}˜7-8 hours) vs. physiologic pH 7.4 (t_{1/2}˜13-25 hours). Only testing at pH 5.0 achieved complete drug release post 24 hours; at pH 7.4˜40% of the drug remained associated with TMV post 72 hours; a plateau is established after ˜2 days thus indicating that longer incubation periods would not achieve further drug release at pH 7.4. The drug release profile of MTO-TMV is similar to other nanoparticle-MTO formulations, e.g., mesoporous silica nanorods. Increased stability under physiologic conditions, e.g., during circulation (pH 7.4), and increased drug release at lower pH (pH 5.0) is expected and desired: under acidic conditions, the carboxylic acid will be protonated thus weakening the charge interactions with MTO triggering its release. This pH dependent release provides favorable conditions for delivery of MTO to the tumor microenvironment; in particular, more aggressive forms of breast cancer such as MDA-MB-231 are known to acidify their surroundings more actively compared to both healthy cells. Furthermore, we previously demonstrated that TMV is taken up by cancer cells including MDA-MB-231 cells. Upon cell uptake, TMV traffics to the endolysosomal compartment where the drug cargo is released and the protein carrier degraded by hydrolysis and proteolysis.

Compared to covalent drug loading strategies, we find the non-covalent drug loading to be more efficient: for example, we have previously demonstrated that doxorubicin can be covalently attached to the interior glutamic acids of TMV using carbodiimide chemistries; however, the maximum drug loading capacity was found to be 270 drug molecules per particle, which is roughly 4× lower than our strategy described here. Furthermore, the drug release mechanism of non-covalent drug delivery systems is favorable as it is markedly faster than drug release in covalently-bound drugs, where the rate-limiting factor for release is the degradation of the particle via hydrolase and protease activity. Given the increased stability of the _{MTO}TMV complex at pH 7.4, mimicking conditions the particles experience during circulation, with t_{1/2} between 13-25 hours, and the rather short circulation half-life of TMV (on the order of minutes), we hypothesize that the non-specific drug release during systemic administration would be minimal.

### _{MTO}TMV Cell Uptake and Cytotoxicity

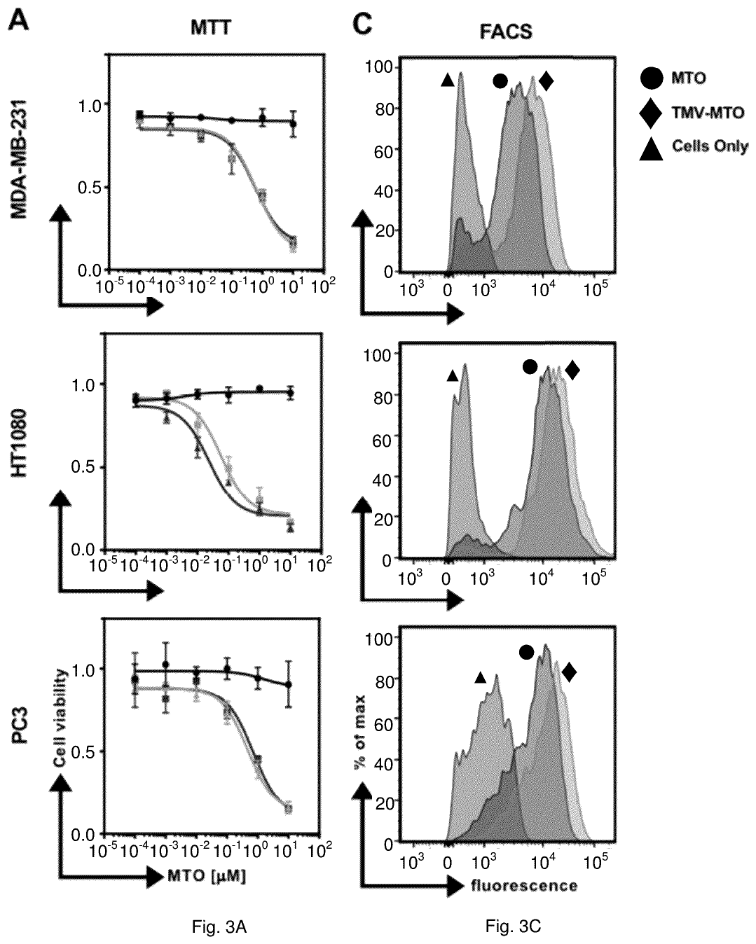

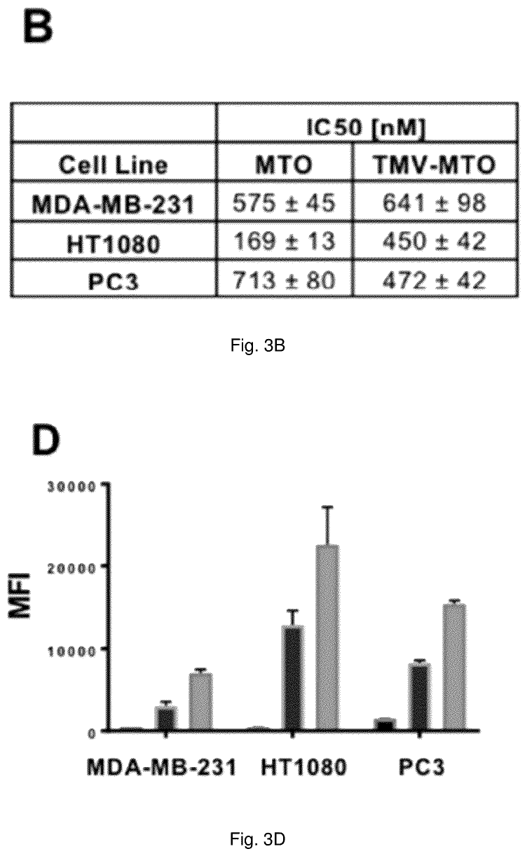

In vitro efficacy of _{MTO}TMV vs. MTO was assessed in a panel of cancer cell lines, including MDA-MB-231 (triple negative breast cancer), HT1080 cells (fibrosarcoma), and PC3 (prostate cancer). The results are consistent in all three cells lines and indicate that _{MTO}TMV retains comparable efficacy compared to its free drug counterpart; IC50 values of the free MTO treatment for MDA-MB-231, HT1080, and PC3 cells were 575±45, 169±13, and 713±80 nM respectively. For the _{MTO}TMV treatment, IC50 values were 641±81, 450±42, and 472±42 nM respectively (FIG. 3B). At the maximum MTO concentrations tested (10 μM), cell viability was suppressed to roughly 20%. Cell uptake of _{MTO}TMV vs. MTO was assessed using flow cytometry taking advantage of the drug's natural fluorescence (Ex/Em 607/684 nm). In each cell line, _{MTO}TMV showed improved cellular uptake compared to MTO. This is reflected by an increase in mean fluorescence intensity (FIG. 3C+D). The increase in cell uptake however does not reflect an increase in cell killing efficiency of the _{MTO}TMV formulation compared to free MTO—this may be explained by slower metabolism of the drug by the tumor cells, due to the added barrier of MTO encapsulation.

Our data are consistent with other reports; drug efficacy is cell line dependent and MTO appears to be more effective in triple negative breast cancer models compared to HER2+ breast cancer subtypes: for example, liposomal MTO and free MTO exhibited IC50 values of 1.25 and 2.13 μM for MTO and encapsulated MTO respectively when delivered to HER2+ MCF-7 breast tumor cells. On the other hand, IC50 values for mesoporous silica nanorods loaded with MTO vs. free MTO against triple negative breast cancer cells MDA-MB-231 lied at 548 and 966 nM, respectively. The latter is in good agreement with our studies. We therefore chose the triple negative model for investigation of drug efficacy using the TMV delivery approach in-vivo.

### In Vivo Drug Delivery Using _{MTO}TMV in a Mouse Model of Triple Negative Breast Cancer

The efficacy of _{MTO}TMV vs. free MTO was assessed in a mouse model of triple negative breast cancer, where MDA-MB-231 xenografts were induced into the subcutaneous space of the right flank of NCR nu/nu mice. Treatment was started when tumors reached 100 mm₃; the treatment schedule comprised three treatments every 5 days of PBS (control), TMV (control), free MTO and _{MTO}TMV at a dose of 1 mg kg₋₁ normalized to MTO (groups were assigned randomly with n=5). Each treatment of _{MTO}TMV was prepared fresh the day of treatment to ensure maximum drug loading and avoiding premature release during extended periods of storage. Tumor burden was measured every other day as a function of tumor volume.

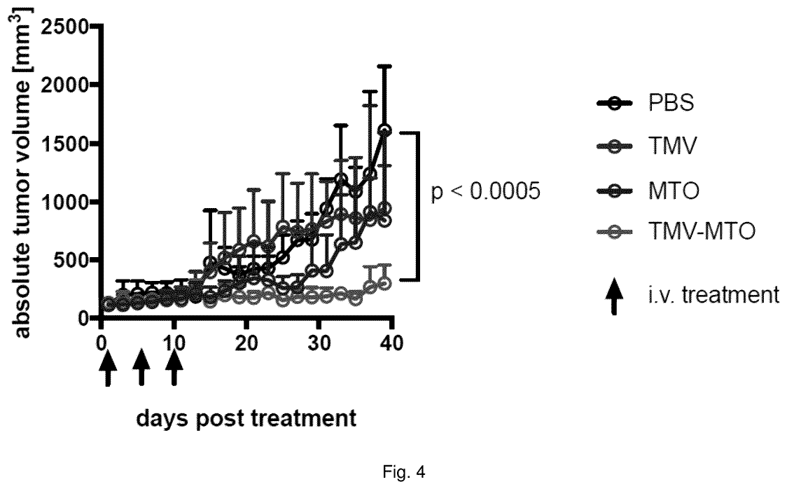

The in-vivo drug delivery study demonstrated that tumor growth rates were significantly suppressed when animals were treated using the _{MTO}TMV formulation: at the endpoint, which was defined as the time point when all PBS-control animals had to be sacrificed based on tumor burden (40 days post first treatment), animals treated with _{MTO}TMV exhibited tumors 5.4× smaller than control tumors (297 mm₃ vs. 1,610 mm₃ for _{MTO}TMV vs. PBS, p<0.0005). There was no statistical significance comparing the PBS vs. TMV vs. MTO groups (FIG. 4).

The enhanced tumor efficacy of _{MTO}TMV vs. free MTO may be explained by the favorable biodistribution of TMV vs. free MTO. In our previous study using phenanthriplatin-loaded TMV and the same MDA-MB-231 mouse model, we found that the amount of drug within the tumors tissue when delivered by TMV was increased by ˜10-fold compared to drug administered systemically. Furthermore, data from our previous biodistribution studies indicate that besides tumor accumulation, TMV is cleared through the liver and spleen with no detectable accumulation in the heart. This biodistribution profile matches other nanotechnologies and is one of the attributes that makes nanocarriers potentially powerful platforms for cancer therapy enabling safer administration of chemotherapy regimens that otherwise would lead to cardiotoxicity.

The complete disclosure of all patents, patent applications, and publications, and electronically available materials cited herein are incorporated by reference. The foregoing detailed description and examples have been given for clarity of understanding only. No unnecessary limitations are to be understood therefrom. In particular, the inventors are not bound by theories described herein. The invention is not limited to the exact details shown and described, for variations obvious to one skilled in the art will be included within the invention defined by the claims.

What is claimed is:

1. A method of treating cancer in a subject, comprising administering to the subject a therapeutically effective amount of an anti-cancer virus particle, the virus particle including a rod-shaped plant virus or virus-like particle and mitoxantrone (MTO) or an analog thereof, wherein the MTO is loaded into the interior channel of the rod-shaped plant virus particle.

2. The method of claim 1, wherein the anti-cancer virus particle is administered to a tumor site in the subject.

3. The method of claim 1, wherein the anti-cancer virus particle is administered to the subject systemically.

4. The method of claim 1, wherein release of the MTO or an analog thereof from the rod-shaped plant virus particle in the subject is pH dependent.

5. The method of claim 4, wherein release is triggered by an acidic tumor microenvironment.

6. The method of claim 1, wherein the cancer is selected from the group consisting of breast cancer, prostate cancer, fibrosarcoma, leukemia and lymphoma.

7. The method of claim 6, wherein the breast cancer is a metastatic breast cancer.

8. The method of claim 7, wherein the breast cancer is triple negative breast cancer.

9. The method of claim 1, further comprising administering a therapeutically effective amount of an additional anticancer agent or therapy to the subject.

10. The method of claim 9, wherein the additional anticancer agent is an antitumor agent.

11. A method of treating triple negative breast cancer in a subject, comprising administering to the subject a therapeutically effective amount of an anti-cancer virus particle, the virus particle including a rod-shaped plant virus or virus-like particle and mitoxantrone (MTO) or an analog thereof, wherein the MTO is loaded into the interior channel of the rod-shaped plant virus particle.

12. The method of claim 11, wherein the anti-cancer virus particle is administered to a triple negative breast cancer tumor site in the subject.

13. The method of claim 11, wherein the anti-cancer virus particle is administered to the subject systemically.

14. The method of claim 11, wherein release of the MTO or an analog thereof from the rod-shaped plant virus particle in the subject is pH dependent.

15. The method of claim 14, wherein release of the MTO or an analog thereof is triggered by an acidic tumor microenvironment.

16. The method of claim 11, further comprising administering a therapeutically effective amount of an additional anticancer agent or therapy to the subject.

17. The method of claim 11, wherein the additional anticancer agent is an antitumor agent.

18. The method of claim 17, wherein the additional anticancer agent is selected from one or more of the group consisting of doxorubicin, vincristine, prednisone adriamycin, Cytoxan, Fluorouracil (5FU), Taxol and Taxotere.

19. The method of claim 17, wherein the additional anticancer therapy includes radiation therapy.

20. The method of claim 17, wherein the additional anticancer therapy includes ablation.
